(19) United States
(12) Patent Application Publication
(10) Pub. No.: US 2017/0119239 A1
(43) Pub. Date: May 4, 2017

(54) DENTAL MIRROR DEVICE

(71) Applicant: Sangse AHN, Seoul (KR)

(72) Inventor: Sangse AHN, Seoul (KR)

(21) Appl. No.: 15/291,425

(22) Filed: Oct. 12, 2016

(30) Foreign Application Priority Data
Nov. 2, 2015 (KR) 10-2015-0153174

(51) Int. Cl. A61B 1/253; A61B 1/015; A61B 1/12; A61B 1/00

(52) U.S. Cl. CPC A61B 1/253; A61B 1/00068; A61B 1/00128; A61B 1/00091; A61B 1/015; A61B 1/126; A61B 1/127

(57) ABSTRACT

In one aspect of the present disclosure, there is provided a dental mirror device comprising: a head having a mirror support to support a mirror thereon; a handle coupled to the head, wherein the handle includes a housing, and a body received in the housing, wherein a first hole is formed in the body; an air sprayer having a hose coupler at a distal end of the handle, an air flow channel formed in the head and handle and air-communicating with the hose coupler, and a sprayer member air-coupled to the air flow channel defined in the head to inject an air to the mirror; and an air flow switch received in the first hole to block or open the air flow channel.

### CROSS-REFERENCE TO RELATED APPLICATION

This application claims the benefit of Korea patent application No. 10-2015-0153174 filed on Nov. 2, 2015, the entire content of which is incorporated herein by reference for all purposes as if fully set forth herein.

### BACKGROUND

Field of the Present Disclosure

The present disclosure relates to a dental mirror device, and, more particularly, to a dental mirror device to allow a clear image on a mirror.

Discussion of Related Art

In order to observe an inner state in an oral cavity, a dental mirror device in a stick form may be used.

A mirror of the dental mirror device may be polluted with contaminants (dust, salvia, dental calculus, drug, etc.) may be fogged due to high temperature and humidity in the oral cavity. Thus, the mirror renders an unclear image thereon.

In order to solve this issue, conventionally, the assistant may manually inject an air into the oral cavity using an air injector. The doctor should wait until the contaminants and fogging are completely removed by the assistant.

When a size of the oral cavity is small as in that of the child, it may be difficult to insert the air injector together with the dental mirror device.

In order to solve the above problems, Korean Patent No. 1241374 discloses a dental mirror device provided to effectively manage the history of patients by storing and outputting information about the oral condition of patients as an image or a video. In this document, a mirror unit includes a mirror to check the oral cavity with the naked eye. The mirror unit includes a heating line to heat the mirror. A housing sprays air to the mirror. The housing includes a vent, an air spray means and an air injection hole. An image information transceiver transmits image information to storage and/or a display device.

However, in the conventional device, the air sprayer should be rotated for the air injection. For this, a power should be fed to the dental mirror device. In this connection, an injected air amount and an injection force may be insufficient to remove the contaminants and fogging on the mirror. The conventional device is heavy.

Because the conventional device needs the power supply, multiple dental treatment chairs should have the multiple dental mirror devices respectively. To solve the problem, the battery is embedded in the device. However, this may require a battery charging.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure is to provide a dental mirror device which does not require a power supply for an air injection and is detachably coupled to an air hose of each dental treatment chair for use.

Further, the present disclosure is to provide a dental mirror device which constantly injects a slight air on the mirror to remove a fogging on the mirror due to a high humidity and temperature in the oral cavity of the patient.

Further, the present disclosure is to provide a dental mirror device which provides a clear image on the mirror from any view angle.

Moreover, the present disclosure is to provide a dental mirror device which is light, is able to be flexible in a movement thereof in a limited space, and has a low maintenance cost.

In one aspect of the present disclosure, there is provided a dental mirror device comprising: a head having a mirror support to support a mirror thereon; a handle coupled to the head, wherein the handle includes a housing, and a body received in the housing, wherein a first hole is formed in the body; an air sprayer having a hose coupler at a distal end of the handle, an air flow channel formed in the head and handle and air-communicating with the hose coupler, and a sprayer member air-coupled to the air flow channel defined in the head to inject an air to the mirror; and an air flow switch received in the first hole to block or open the air flow channel.

In one implementation, the air flow switch comprises: a cylinder having an air inlet defined therein communicating with the air flow channel defined in the handle, an air outlet defined therein communicating with the air flow channel defined in the head, and an air flow switching space defined between the air inlet and air outlet to air-communicate with the air inlet and air outlet; and a vertical-movable valve received in the cylinder to open or close the air flow switching space via a vertical movement thereof.

In one implementation, the cylinder has the air-communication groove formed in and along an outer face of the cylinder to ensure a constant air-communication between the air inlet and air outlet, wherein the air-communication groove is sized such that an air flow rate through the air-communication groove is smaller than an air flow rate through the air flow switching space.

In one implementation, the dental mirror device further comprises a coupler to allow attachment/detachment of the head to and from the handle, wherein the coupler includes a second hole, a coupling protrusion and an elastic member, wherein the second hole is defined at an overlapping position between the head and handle, wherein the coupling protrusion is inserted into the second hole and is protruded through the second hole out of the second hole, wherein the elastic member is disposed beneath the coupling protrusion to push upward the coupling protrusion to allow the coupling protrusion to protrude through the second hole out of the second hole.

In one implementation, the sprayer member has a main nozzle with a shortest extension, and auxiliary nozzles branching from the main nozzle, wherein the auxiliary nozzles are arranged symmetrically relative to the main nozzle.

In one implementation, the vertical-movable valve of the air flow switch includes a push plate, a first vertical-movable member, a second vertical-movable member, a returning member and a sealing ring, wherein the push plate is configured to be pushed up or down through the first hole defined in the body, wherein the first vertical-movable member is disposed beneath the push plate, wherein the second vertical-movable member is disposed beneath the first vertical-movable member to contact the first vertical-movable member, wherein the returning member is disposed beneath the second vertical-movable member to push up the second vertical-movable member, wherein the sealing ring is disposed on the second vertical-movable member to air-tightly seal the air flow switching space in the cylinder.

In accordance with the present disclosure, the dental mirror device does not require a power supply for an air injection and is detachably coupled to an air hose of each dental treatment chair for use.

Further, in accordance with the present disclosure, the dental mirror device constantly injects a slight air on the mirror to remove a fogging on the mirror due to a high humidity and temperature in the oral cavity of the patient.

Further, in accordance with the present disclosure, the dental mirror device provides a clear image on the mirror from any view angle.

Moreover, in accordance with the present disclosure, the dental mirror device is light, is able to be flexible in a movement thereof in a limited space, and has a low maintenance cost.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 is a perspective view of a dental mirror device in accordance with the present disclosure.

FIG. 2 is an exploded perspective view of a dental mirror device in accordance with the present disclosure.

FIG. 3 show a cross sectional view of an air flow switch in accordance with the present disclosure.

FIG. 4 illustrates an operation of the air flow switch.

FIG. 5 illustrates a detailed configuration of a sprayer member

FIG. 6 illustrates a detailed configuration of the air-communication groove.

For simplicity and clarity of illustration, elements in the figures are not necessarily drawn to scale. The same reference numbers in different figures denote the same or similar elements, and as such perform similar functionality. Also, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

### DETAILED DESCRIPTIONS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

It will be understood that when an element or layer is referred to as being “connected to”, or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element s or feature s as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented for example, rotated 90 degrees or at other orientations, and the spatially relative descriptors used herein should be interpreted accordingly.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, s, operations, elements, and/or components, but do not preclude the presence or addition of one or more other features, integers, s, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description, numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.”

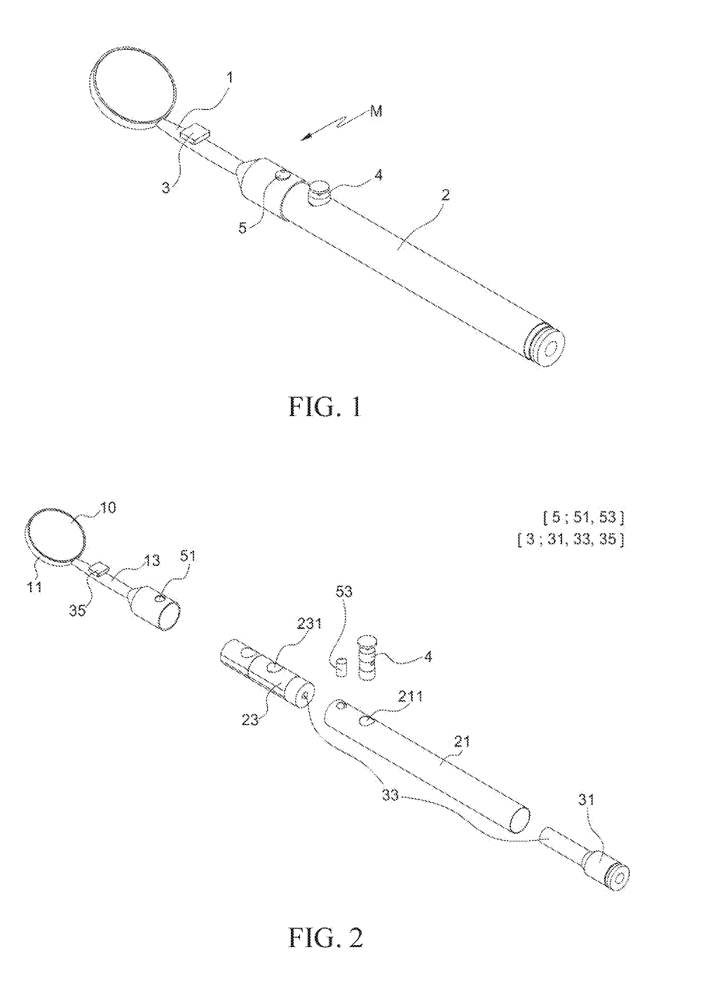

FIG. 1 is a perspective view of a dental mirror device in accordance with the present disclosure. FIG. 2 is an exploded perspective view of a dental mirror device in accordance with the present disclosure.

As shown in FIG. 1 and FIG. 2, the present dental mirror device M may include a head 1, a handle 2, an air sprayer 3 and an air flow switch 4.

Referring to FIG. 1 and FIG. 2, a head 1 may have a mirror support 11 to support a mirror 10 thereon. The mirror support 11 may be made of a magnetic or ferromagnetic material. The mirror 10 may be made of a magnetic or ferromagnetic material.

The mirror 10 may be magnetically attached to or detached from the mirror support 11. In this way, using a ferromagnetic material-containing object, the mirror 10 may be replaced easily.

In addition, the mirror 10 may have a coating thereon to improve a light reflection or provide a clear image.

Further, as shown in FIG. 1 and FIG. 2, the head 1 has a body 13 being tapered toward the mirror 10.

This tapering feature may improve an air injection force. Further, the feature may remedy a space limitation together with the support 11 being thin. For example, when dealing with a child with a small oral cavity, this feature may allow the dental doctor to move the present device freely in the relatively narrow oral cavity.

The mirror support 11 may have a thickness of 1 mm to 5 m, more preferably, 3 mm.

Further, in order to remedy the space limitation, the mirror support 11 and the body 13 may be coupled in an angled manner to allow the mirror support 11 to extend in a tiled or upward direction.

For the sake of convenience, the present dental mirror device M may be small and light. For this, each component thereof may be small and light.

The head 1 may be coupled to the handle 2. The handle 2 may be gripped by the user. The handle 2 may include a housing 21 and a body 23 received in the housing 21.

As shown in FIG. 1 and FIG. 2, the body 23 may have a first hole 231 defined therein, in which the air flow switch 4 may be inserted.

Further, the housing 21 may have a second hole 211 defined therein to position-overlap the first hole 231. Through the second hole 211, a push plate 430 of the air flow switch 4 may protrude out of the second hole 211.

As shown in FIG. 1 and FIG. 2, the head 1 may be detached from and attached to the handle 2 via a coupler 5. The coupler 5 may include a third hole 51, a coupling protrusion 53 and an elastic member (not shown).

To be specific, the third hole 51 may be defined in the head 1. When the head and handle are coupled to each other, the third hole 51 may be placed at an overlapping position thereof.

The coupling protrusion 53 may be included in the handle 2. The coupling protrusion 53 may be inserted into the third hole 51 and may be protruded through the third hole 51 out of the hole 51. The coupling protrusion 53 may be placed at an overlapping position of the head and handle.

The elastic member (not shown) may be disposed beneath the coupling protrusion 53 to push upward the coupling protrusion 53 to allow the coupling protrusion 53 to protrude through the third hole 51 out of the hole 51.

The present dental mirror device M may be divided into the head 1 and handle 2 by pushing the coupling protrusion 53 down and into the third hole 51. When the head 1 and handle 2 are coupled to each other, the coupling protrusion 53 is protruded through the third hole 51 out of the hole 51 to stop the head 1 and handle 2.

When the present dental mirror device M is divided into the head 1 and handle 2 by pushing the coupling protrusion 53 down, the head 1 may be cleaned or sterilized.

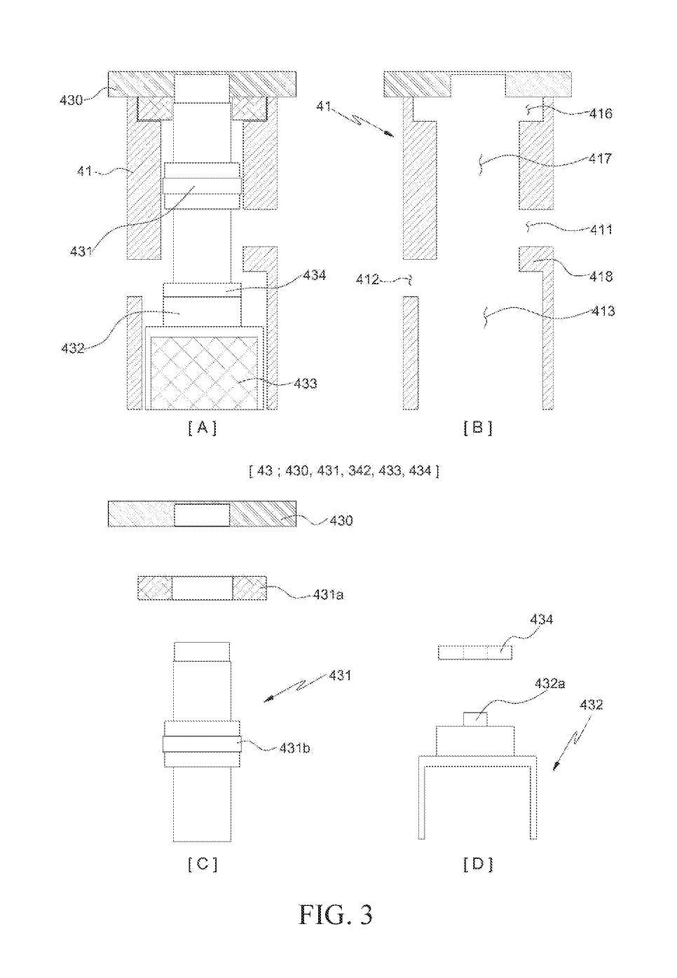

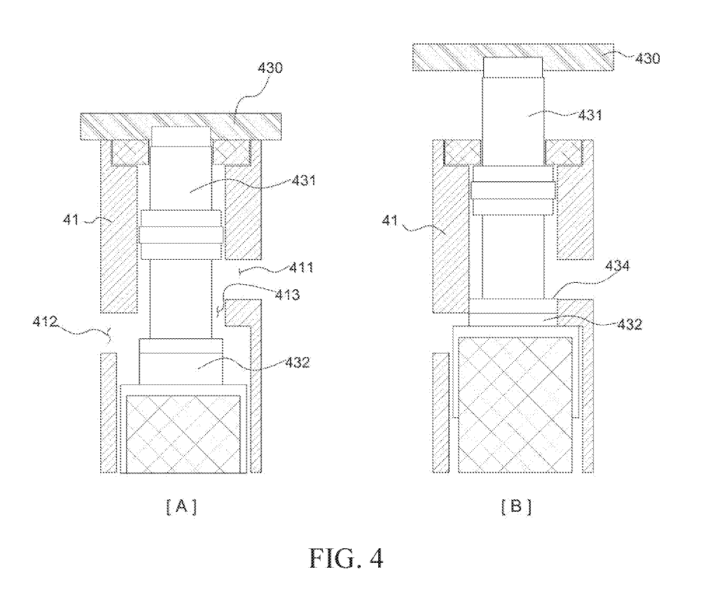

FIG. 3 show a cross sectional view of an air flow switch in accordance with the present disclosure.

As shown in FIG. 1 to FIG. 3, the air sprayer 3 may inject an air via an air hose to a mirror 10 to remove a contaminant or fogging on the mirror 10. The air sprayer 3 may include a hose coupler 31, an air flow channel 33 and a sprayer member 35.

The hose coupler 31 may be disposed in a rear end of the handle 2 to be coupled to the air hose.

The air flow channel 33 may be defined in and along the head 1 and handle 2 to be communicated with the hose coupler 31 to receive an air from the air hose.

The sprayer member 35 may be disposed on the head 1 and be communicated with the air flow channel 33 defined in the head 1 to inject the air to the mirror 10.

The air sprayer 3 may be configured to receive an air via the hose coupler 31 from the air hose coupled to an air compressor installed in the dental clinic. The air may flow along the air flow channel 33. The sprayer member 35 may inject an air to the mirror 10 to remove a contaminant or fogging on the mirror 10.

As shown in FIG. 1 to FIG. 3, especially, in FIG. 3, the air flow switch 4 may be configured to control whether to inject an air to the mirror 10. The air flow switch 4 may include a cylinder 41 mounted into the first hole 231, and a vertical-movable valve 43 embedded in the cylinder 41.

The cylinder 41 may have an air inlet 411 defined therein communicating with the air flow channel 33 defined in the handle 2, an air outlet 412 defined therein communicating with the air flow channel 33 defined in the head 1, and an air flow switching space 413 between the air inlet 411 and air outlet 412 to communicate with the air inlet 411 and air outlet 412.

The vertical-movable valve 43 may be configured to open or close the air flow switching space 413.

The air flow switch 4 may be configured to control whether to inject an air via the sprayer member 35 by opening or closing the air flow switching space 413 defined in the cylinder 41 during an air flow along the air flow channel 33.

The present dental mirror device M may use the air sprayer 3 to inject an air to a mirror 10 to remove a contaminant or fogging on the mirror 10 to ensure a clear image thereon. Conventionally, to remove a contaminant or fogging on the mirror 10, an assistant should manually inject an air into the oral cavity of the patient. Using the present dental mirror device M, the doctor may turn on the air flow switch 4 to inject the air to the mirror 10 when he/she wants to.

The present dental mirror device M may be air-coupled to the air compressor installed in the dental clinic. Thus, the doctor may hold a single present dental mirror device M and move between multiple dental treatment chairs. Thus, an individual dental mirror device may not be required for each of the multiple dental treatment chairs.

FIG. 3 illustrates the more specific configuration of the air flow switch 4.

As shown in FIG. 3, the vertical-movable valve 43 of the air flow switch 4 may include the push plate 430, a first vertical-movable member 431, a second vertical-movable member 432, a returning member 433 and a sealing ring 434.

Referring to FIG. 3, the push plate 430 may be configured to be pushed up or down through the second hole 211 defined in the housing 21. The user may push down the push plate 430 to remove the contaminant or fogging on the mirror 10.

The first vertical-movable member 431 may be disposed beneath the push plate 430 and be fixed to the push plate 430. Thus, when the push plate 430 is pushed down, the first vertical-movable member 431 may be lowered down in the cylinder 41.

The second vertical-movable member 432 may be disposed beneath the first vertical-movable member 431 to contact the first vertical-movable member 431. When the push plate 430 is pushed down, and, thus, the first vertical-movable member 431 is lowered down in the cylinder 41, the second vertical-movable member 432 may be lowered down in the cylinder 41.

The returning member 433 may be disposed beneath the second vertical-movable member 432 to push up the second vertical-movable member 432. When the pressure on the push plate 430 is removed, the returning member 433 may push up the second vertical-movable member 432, which may push the first vertical-movable member 431 and push plate 430 up together.

The sealing ring 434 may be disposed on the second vertical-movable member 432 to air-tightly seal the air flow switching space 413 in the cylinder 41. To this end, the sealing ring 434 may have the diameter as that of the second vertical-movable member 432 and as an inner diameter of the air flow switching space 413.

As shown in FIG. 3B, the cylinder 41 may further have, in addition to the air inlet 411, the air outlet 412, and the air flow switching space 413, as described above, a shoulder 416, an upper space 417 and a stopper 418.

The shoulder 416 may allow a ring member 431a to be seated thereon. The ring member 431a may wrap the first vertical-movable member 431.

The upper space 417 may allow the first vertical-movable member 431 and second vertical-movable member 432 to vertically move therein and therealong.

The stopper 418 may be configured to stop the second vertical-movable member 432 at a predetermined level when the second vertical-movable member 432 is pushed up by the returning member 433. In this way, the first vertical-movable member 431 and second vertical-movable member 432 may be prevented from movement out of the cylinder 41.

As shown in FIG. 3C, the first vertical-movable member 431 may further have the ring member 431a wrapping an upper portion of the first vertical-movable member 431, and an O-ring 431b wrapping a middle portion of the first vertical-movable member 431.

The ring member 431a may act to tightly seal the second hole 211 through which the push plate 430 protrudes upwards.

The O-ring 431b may act to improve sealing effect of the cylinder 41. The O-ring 431b may have an elastic property and thus contact an upper inner wall of the cylinder 41 in a deformed manner. The deformation of the O-ring 431b may be set not to disable the movement of the first vertical-movable member 431 in the upper space 417.

As shown in FIG. 3D, the second vertical-movable member 432 may have a coupler 432a protruding upwardly therefrom. The coupler 432a has a smaller diameter than that of the second vertical-movable member 432. The coupler 432a may be fitted with the sealing ring 434.

FIG. 4 illustrates an operation of the air flow switch.

As shown in FIG. 4 A, the push plate 430 may be pushed down to move the first vertical-movable member 431 and second vertical-movable member 432 down. Thus, the air flow switching space 413 of the cylinder 41 may be open, and, hence, the air inlet 411 and air outlet 412 may be air-communicated with each other. Thus, the air from the air hose may be delivered to the sprayer member 35 which may inject the air to the mirror.

As shown in FIG. 4B, the returning member 433 may push up the first vertical-movable member 431 and second vertical-movable member 432. Thus, the air flow switching space 413 of the cylinder 41 may be closed, and, hence, the air inlet 411 and air outlet 412 may be air-blocked with each other. Thus, the supply of the air from the air hose to the sprayer member 35 may be blocked. Thus, injecting the air to the mirror may be disabled.

In this way, under control of the air flow switch 4, the air sprayer 3 injecting the air to the mirror may be disabled or enabled.

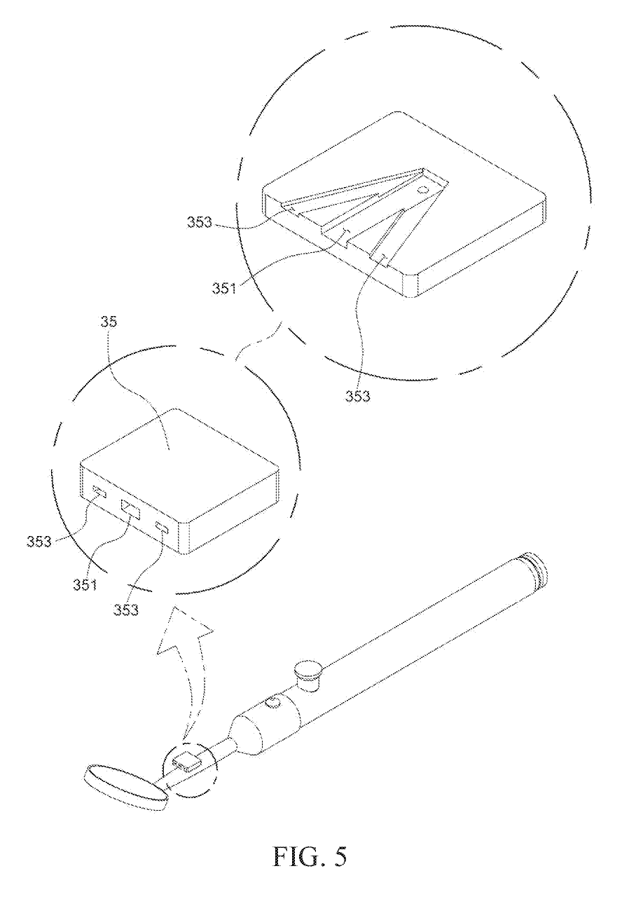

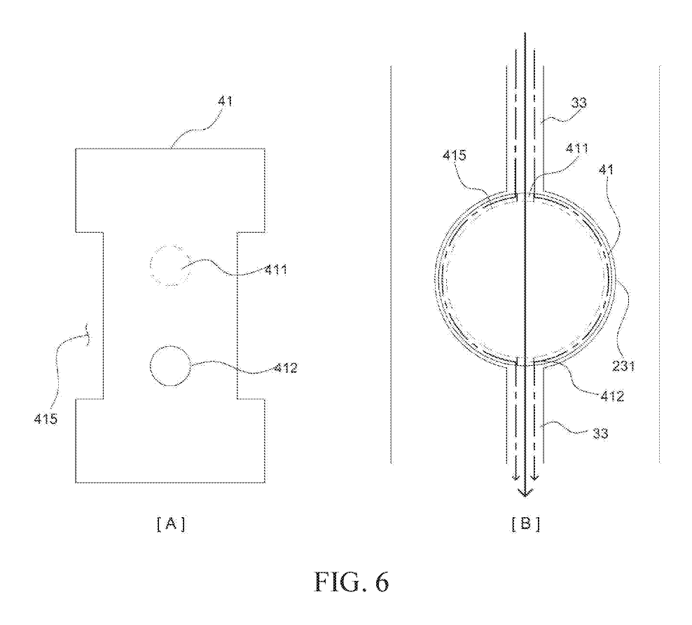

Now, with reference to FIG. 5 and FIG. 6, further features of the present disclosure will be described.

FIG. 5 illustrates a detailed configuration of the sprayer member. As shown in FIG. 5, the sprayer member 35 may have a main nozzle 351 having a shortest length, and auxiliary nozzles 353 branching from the main nozzle 351.

The main nozzle 351 and auxiliary nozzles 353 may allow a uniform spread of the air across the mirror 10, thereby to more reliably remove the contaminants and fogging.

As shown in FIG. 5, a width of the main nozzle 351 may be larger than that of each of the auxiliary nozzles 353. This feature may exhibit following two effects: first, a pressure of an air injected from the main nozzle 351 may be equal to a pressure of an air injected from the auxiliary nozzles 353, which, otherwise, may be lower than the pressure of an air injected from the main nozzle 351. In this way, injected air-beam pressures may be uniform across the surface of the mirror. Second, the larger amount of the air injected from the main nozzle 351 may lead to the fact that the injected air into the center of the mirror 10 may push the contaminants and fogging from the center to an outer perimeter. From such effects, the present dental mirror device may allow a clear image on the mirror 10.

FIG. 6 illustrates a detailed configuration of the air-communication groove. As shown in FIG. 6, the air-communication groove 415 may be formed in and along an outer face of the cylinder 41 to ensure a constant air-communication between the air inlet 411 and air outlet 412.

The oral cavity of the patient may have a high temperature and humidity. Thus, a fogging may be formed on the mirror 10. In order to prevent the fogging, the air-communication groove 415 may allow the constant air injection to the mirror 10 regardless of the air injection activated upon pushing down the switch 4.

The air flow rate through the air-communication groove 415 may be small enough to suppress the fogging on the mirror 10. The air-communication groove 415 may be sized such that the air flow rate through the air-communication groove 415 is smaller than the air flow rate through the air flow switching space 413.

As shown in FIG. 6B, the air flow through the air flow switching space 413 and the air flow through the air-communication groove 415 may be indicated by different arrows. The size of the air-communication groove 415 may be selected by the skilled person to the art as appropriate.

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims. Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

What is claimed is:

1. A dental mirror device comprising:
a head having a mirror support to support a mirror thereon;
a handle coupled to the head, wherein the handle includes a housing, and a body received in the housing, wherein a first hole is formed in the body;
an air sprayer having a hose coupler at a distal end of the handle, an air flow channel formed in the head and handle and air-communicating with the hose coupler, and a sprayer member air-coupled to the air flow channel defined in the head to inject an air to the mirror; and
an air flow switch received in the first hole to block or open the air flow channel,
wherein the air flow switch comprises:
a cylinder having an air inlet defined therein communicating with the air flow channel defined in the handle, an air outlet defined therein communicating with the air flow channel defined in the head, and an air flow switching space defined between the air inlet and air outlet to air-communicate with the air inlet and air outlet; and
a vertical-movable valve received in the cylinder to open or close the air flow switching space via a vertical movement thereof,
wherein the cylinder has the air-communication groove formed in and along an outer face of the cylinder to ensure a constant air-communication between the air inlet and air outlet,
wherein the air-communication groove is sized such that an air flow rate through the air-communication groove is smaller than an air flow rate through the air flow switching space.

2. The dental mirror device of claim 1, further comprising a coupler to allow attachment/detachment of the head to and from the handle,
wherein the coupler includes a second hole, a coupling protrusion and an elastic member,
wherein the second hole is defined at an overlapping position between the head and handle,
wherein the coupling protrusion is inserted into the second hole and is protruded through the second hole out of the second hole,
wherein the elastic member is disposed beneath the coupling protrusion to push upward the coupling protrusion to allow the coupling protrusion to protrude through the second hole out of the second hole.

3. The dental mirror device of claim 1, wherein the sprayer member has a main nozzle with a shortest extension, and auxiliary nozzles branching from the main nozzle, wherein the auxiliary nozzles are arranged symmetrically relative to the main nozzle.

4. The dental mirror device of claim 1, wherein the vertical-movable valve of the air flow switch includes a push plate, a first vertical-movable member, a second vertical-movable member, a returning member and a sealing ring,
wherein the push plate is configured to be pushed up or down through the first hole defined in the body,
wherein the first vertical-movable member is disposed beneath the push plate,
wherein the second vertical-movable member is disposed beneath the first vertical-movable member to contact the first vertical-movable member,
wherein the returning member is disposed beneath the second vertical-movable member to push up the second vertical-movable member,
wherein the sealing ring is disposed on the second vertical-movable member to air-tightly seal the air flow switching space in the cylinder.
